(19) United States
(12) Patent Application Publication
(10) Pub. No.: US 2004/0138824 A1
(43) Pub. Date: Jul. 15, 2004

(54) Linkage analysis using direct and indirect counting

(76) Inventors: Yang Da, Maplewood, MN (US); John R. Garbe, Roseville, MN (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/340,286

(22) Filed: Jan. 9, 2003

(51) Int. Cl. C12Q 1/68; G06F 19/00; G01N 33/48; G01N 33/50

(52) U.S. Cl. 702/20; 435/6

(57) ABSTRACT

A method based on direct and indirect counting is disclosed for rapid and accurate linkage analysis for codominant and dominant loci. Methods for estimating gender-specific recombination frequencies are available for cases where at least one of the two loci is multi-allelic and for bi-allelic loci with mixed parental linkage phases where at least one locus is codominant. The method makes use of the full data set, yields exact estimates of the recombination frequencies when the observed and expected genotypic frequencies are equal, and are computationally efficient.

### STATEMENT OF GOVERNMENT RIGHTS

The present invention was made, at least in part, with a grant from the Government of the United States of America (NRICGP/USDA grant# 03275). The Government may have certain rights to the invention.

### FIELD

The present invention relates generally to performing genetic linkage analysis, and more particularly to linkage analysis using indirect counting methods.

### COPYRIGHT NOTICE/PERMISSION

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever. The following notice applies to the software and data as described below and in the drawings hereto: Copyright ® 2002, Regents of the University of Minnesota, All Rights Reserved.

### BACKGROUND

Genetic linkage analysis is a statistical method that is used to associate functionality of genes to their location on chromosomes. It is based on the observation that genes that reside physically close on a chromosome remain linked during meiosis. Typically, markers which are found in vicinity on the chromosome have a tendency to stick together when passed on to offspring. Thus, if some disease is often passed to offspring along with specific markers, then it can be concluded that the gene(s) which are responsible for the disease are located close on the chromosome to these markers.

Genetic linkage is designed to estimate the distance between genes. Normally, immediately before the gametes (sperm or eggs) are produced, there is a lining up of parental chromosomes in preparation for the separation of genetic material into gametes. An exchange of genetic material occurs between parental chromosomal pairs, which is termed recombination, or crossing over between chromosomes. The chromosomes are then separated and packaged into the gametes.

Two genes that lie on separate chromosomes will be transmitted independently of each other from parent to child. The child has an equal chance of receiving the gene from his mother or from his father. This phenomenon is encapsulated in Mendel's law of independent assortment.

However, two genes may also be on the same chromosome. If they are located at opposite ends, then they will once again be transmitted independently of each other. This is because they are so far away from each other that a recombination event is very likely to occur between the two loci. However, the closer the two genes lie to each other, the less likely it is that a genetic crossover will occur between them. Finally, two genes may lie so close that it is much more likely that they will remain together and be transmitted together into the forming gamete. Two examples make this clearer.

If an individual has genotype A1A2 at locus A and genotype B1B2 at locus B and the loci are not linked to each other, the alleles at locus A and locus B will assort independently and four different types of gametes (A1B1, A1B2, A2B1, A2B2) will be produced in equal frequencies. This is termed independent assortment.

If locus A is very close to locus B on the same chromosome, an individual will again produce four types of gametes, but now the alleles found will not be in equal frequencies. The most common types of gametes will be those that represent the alleles that occurred in each parent. The less frequent types of gametes will contain a mixture of the parental alleles that has occurred as a result of infrequent recombination events between the two loci.

While computationally efficient methods are available for large scale linkage analysis for codominant loci, rapid methods are unavailable for mapping dominant loci and for the map integration of dominant and codominant loci. Most computer programs that provide linkage analysis for dominant loci such as LINKAGE implement computationally intensive likelihood analysis and generally have a limitation on the number of loci that can be analyzed jointly. A computationally efficient method for linkage analysis with codominant and dominant inheritance is needed for mapping dominant genes and for the map integration of codominant and dominant loci, because dominant inheritance mode is typical of many disease genes and many dominant markers (such as RAPD and AFLP markers) exist. Analytical formulas for maximum likelihood estimate of recombination frequency between two dominant loci in repulsion linkage phase have been developed. However, the mathematical simplicity of such an analytical formula is computationally efficient for large scale linkage analysis. However, many other cases of linkage analysis do not have a simple analytical formula for estimating recombination frequencies. The understanding of relative efficiencies of various types of genotypic data is useful for planning mapping experiments. Most results on relative efficiencies of genotypic data were based on the approximate variances and covariances of estimated recombination frequencies but the accuracy of such an approximation is unclear.

Additionally, sex-influenced traits can affect linkage analysis. A sex-influenced trait has an autosomal inheritance mode that typically exhibits the pattern of “reversal dominance” in the two genders, i.e., the gene is dominant in one gender and recessive in the other. Examples of sex-influenced traits have been reported in several species. Scurs of cattle requires one scurred allele to express in males and two scurred alleles to express in females. The depth of the red color of the Ayrshire cattle is dominant in males and recessive in females. A gene affecting a chicken plumage pattern is dominant in males and recessive in females. Human baldness and short index fingers are dominant in men and recessive in women, whereas the disorder of Heberden nodes, which are bony excrescences of the phalanges of the distal interphalangeal joints of the fingers, is likely to be dominant in women and recessive in men. Another human example is the inheritance of one form of Aarskog's faciodigitogenital syndrome. Furthermore, it was recently conjectured that factors affecting the development of rheumatoid arthritis in humans show sex-influenced expression. Examples of sex-influenced traits have also been observed in mice and insects. Although methods are available for linkage analysis, a method for linkage analysis involving a sex-influenced gene is unavailable in conventional linkage analysis systems.

In view of the problems discussed above, there is a need in the art for the present invention.

### SUMMARY

The above-mentioned shortcomings, disadvantages and problems are addressed by the present invention, which will be understood by reading and studying the following specification.

The present invention includes systems and methods for analyzing genetic data using direct and indirect counting. One aspect of the present invention includes systems and methods that receive input data including family identification and genetic identifiers and extracting statistics regarding the genetic identifiers. The statistics may be used to compute at least one recombination frequency and LOD score for at least one locus by applying indirect counting to the statistics. In addition, the systems and methods may use the recombination frequencies and LOD scores to determining a locus order for the genetic identifiers.

A further aspect of the present invention is that inheritance cases are determined that then may be used to determine an appropriate indirect counting solution.

A still further aspect is that the indirect counting solution may use iterative computation to arrive at a recombination frequency.

The present invention describes systems, clients, servers, methods, and computer-readable media of varying scope. In addition to the aspects and advantages of the present invention described in this summary, further aspects and advantages of the invention will become apparent by reference to the drawings and by reading the detailed description that follows.

### BRIEF DESCRIPTION OF THE DRAWINGS

FIG. 1 is a block diagram of a software operating environment for performing linkage analysis in which different embodiments of the invention may be practiced;

FIGS. 2A-2C are diagrams providing further details of input files used in the software operating environment;

FIG. 3 is a diagram providing further details of screen output provided by the software operating environment;

FIGS. 4A-4E are diagrams providing further details of output files provided by the software operating environment;

FIGS. 5A-5E are flowcharts illustrating methods for performing linkage analysis using direct and indirect counting; and

FIG. 6 is a diagram illustrating the major hardware components of a computer incorporating embodiments of the invention.

### DETAILED DESCRIPTION

In the following detailed description of exemplary embodiments of the invention, reference is made to the accompanying drawings which form a part hereof, and in which is shown by way of illustration specific exemplary embodiments in which the invention may be practiced. These embodiments are described in sufficient detail to enable those skilled in the art to practice the invention, and it is to be understood that other embodiments may be utilized and that logical, mechanical, electrical and other changes may be made without departing from the scope of the present invention.

Some portions of the detailed descriptions which follow are presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like. It should be borne in mind, however, that all of these and similar terms are to be associated with the appropriate physical quantities and are merely convenient labels applied to these quantities. Unless specifically stated otherwise as apparent from the following discussions, terms such as “processing” or “computing” or “calculating” or “determining” or “displaying” or the like, refer to the action and processes of a computer system, or similar computing device, that manipulates and transforms data represented as physical (e.g., electronic) quantities within the computer system's registers and memories into other data similarly represented as physical quantities within the computer system memories or registers or other such information storage, transmission or display devices.

In the Figures, the same reference number is used throughout to refer to an identical component which appears in multiple Figures. Signals and connections may be referred to by the same reference number or label, and the actual meaning will be clear from its use in the context of the description.

The following detailed description is, therefore, not to be taken in a limiting sense, and the scope of the present invention is defined only by the appended claims.

### Operating Environment

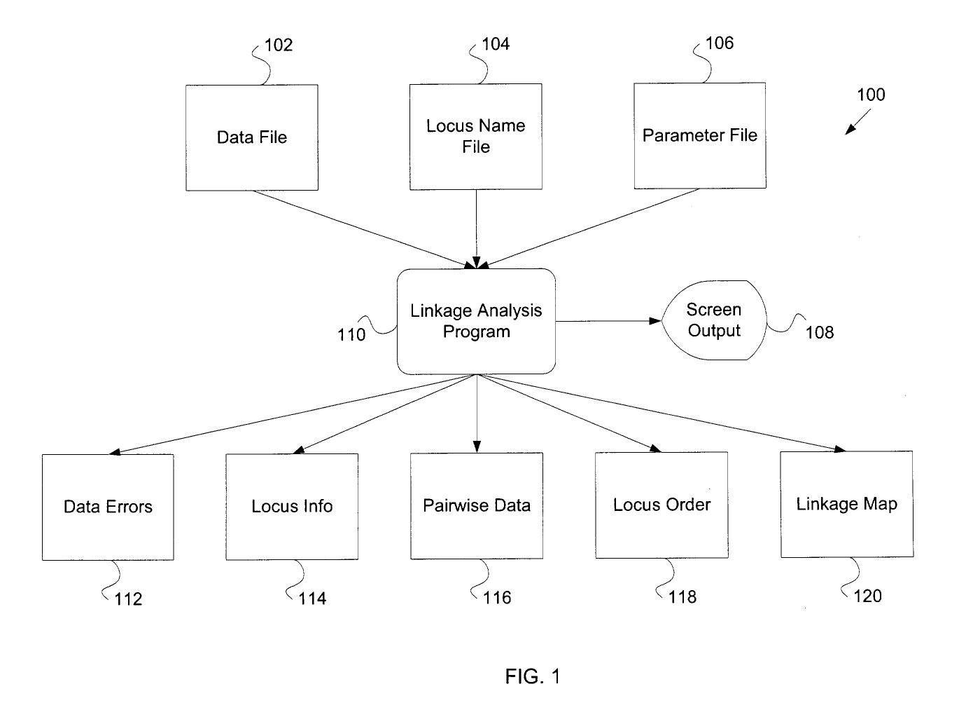

FIG. 1 is a block diagram of a software operating environment 100 for performing linkage analysis in which different embodiments of the invention may be practiced. In some embodiments of the invention, software environment 100 includes a linkage analysis program 110 that receives input from data file 102, name file 104 and parameter file 106. Note that while three input files may be used in some embodiments, the data in the files could be provided in other combinations of one or more input files or data streams. In one embodiment of the invention, linkage analysis program 110 is the Locusmap program available from the University of Minnesota. In some embodiments, linkage analysis program 110 uses the input data provided in files 102, 104 and 106 and the methods described in further detail below to produce screen output 108, data errors 112, locus info data 114, pairwise data 116, locus order data 118 and linkage map 120.

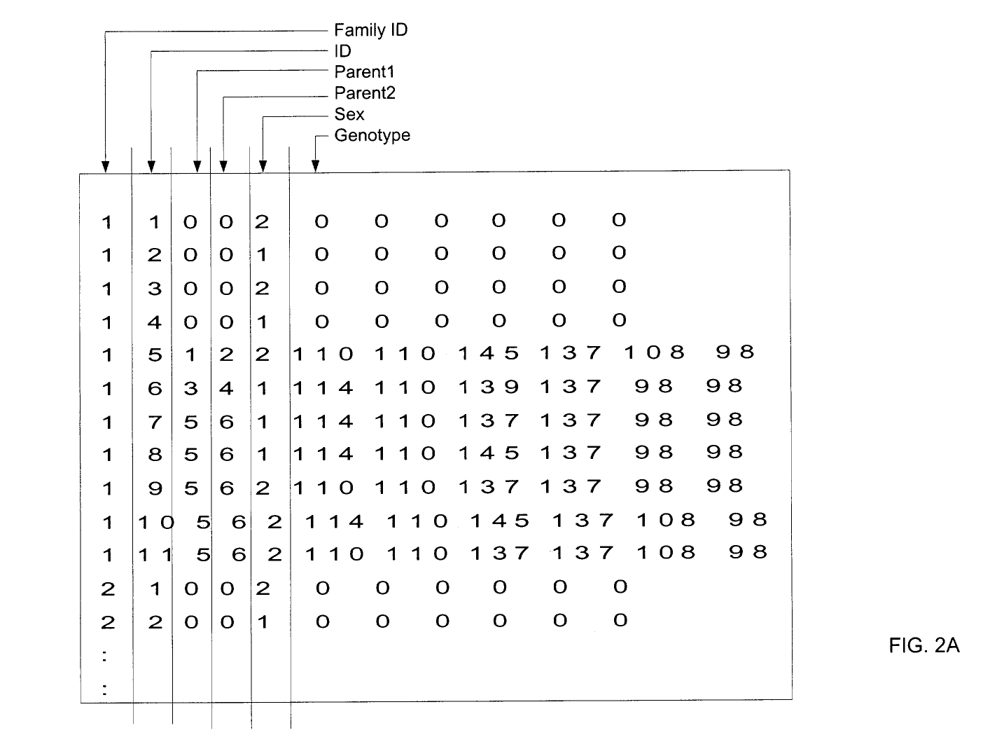

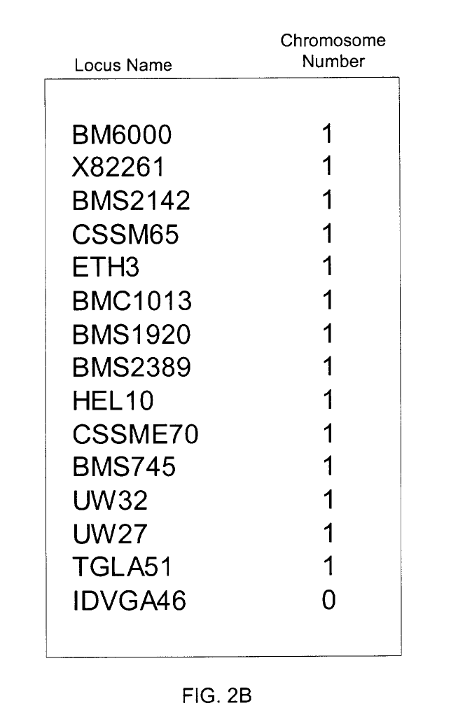

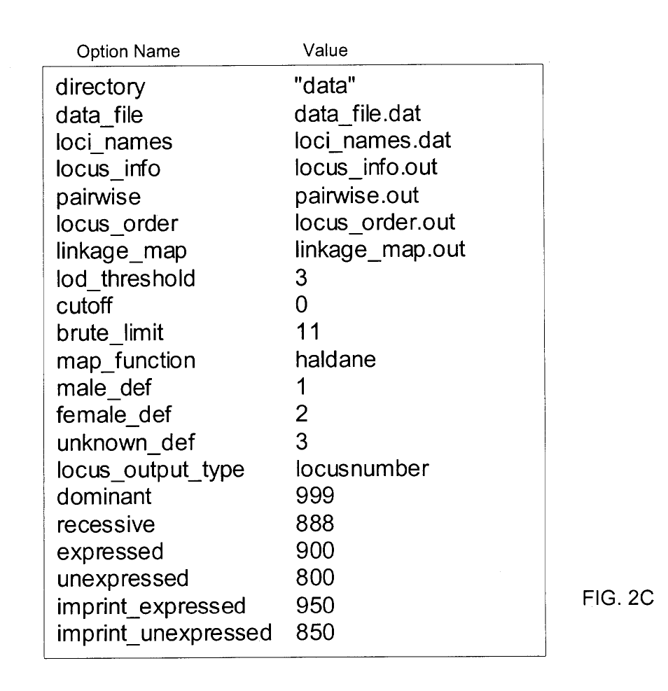

FIG. 2A is a diagram providing details of the information in data file 102. As illustrated in FIG. 2A, data file 102 in some embodiments of the invention has data for a number of individuals in a number of different families. The data for each individual may include various combinations of the following:

Family ID—Identifies a family to which the individual belongs.

ID—Uniquely identifies the individual.

Parent 1—ID for a parent of the individual.

Parent 2—ID for a second parent of the individual.

Sex—Gender of the individual.

Genotype—one or more pairs of alleles forming loci. Phenotype information may also be included in some embodiments.

Although the various values in FIG. 2A are numeric, those of skill in the art will appreciate that other non-numeric data could be substituted.

FIG. 2B is a diagram providing details of the information in name file 104. In some embodiments, the name file provides a mapping between a locus name and a chromosome number.

FIG. 2C is a diagram providing details of the information in parameter file 106. In some embodiments of the invention, parameter file 106 includes data providing the name and expected location of various input and output files. Further, the parameter file may include encoding values for gender and traits. In addition, in some embodiments of the invention, parameter file 106 includes various combinations of the following parameters:

lod_threshold—LOD (logarithm of odds) score value used to determine if linkage is present.

cutoff—the minimum number of offspring in a phase unknown family in order for the family to be used in calculations.

brute_limit—maximum number of loci to use brute-force ordering.

map_function—function used to convert recombination frequency to a genetic distance. Values include Haldane, Morgan and Kosambi.

Locus_output_type—determines whether locus name or number are output.

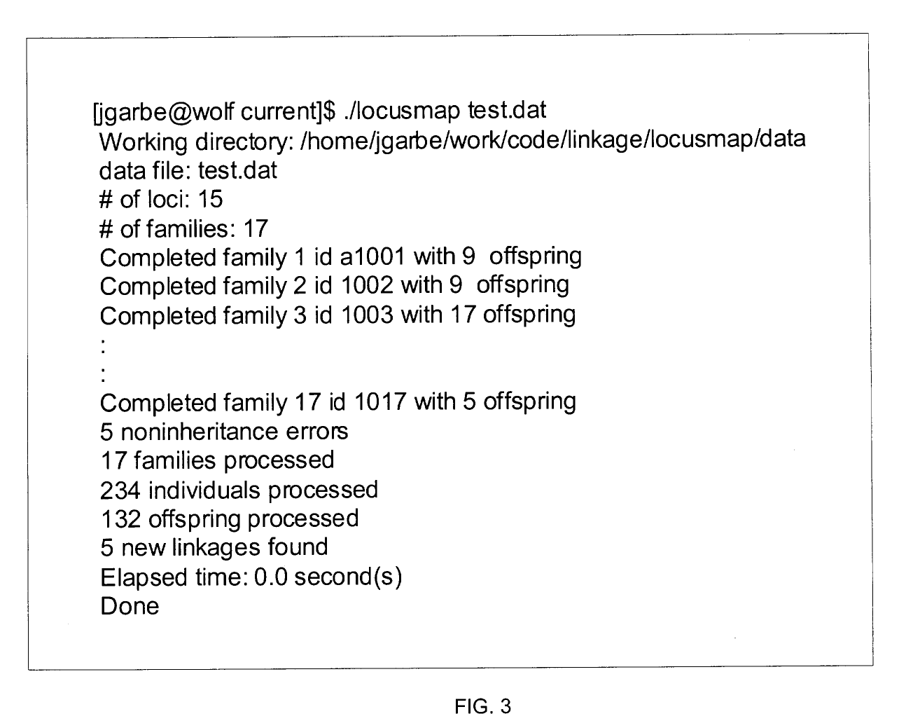

FIG. 3 is a diagram providing further details of screen output 108 provided by linkage analysis program 110. Screen output is not required, but may be useful to determine the progress of the linkage analysis program and whether errors are being encountered.

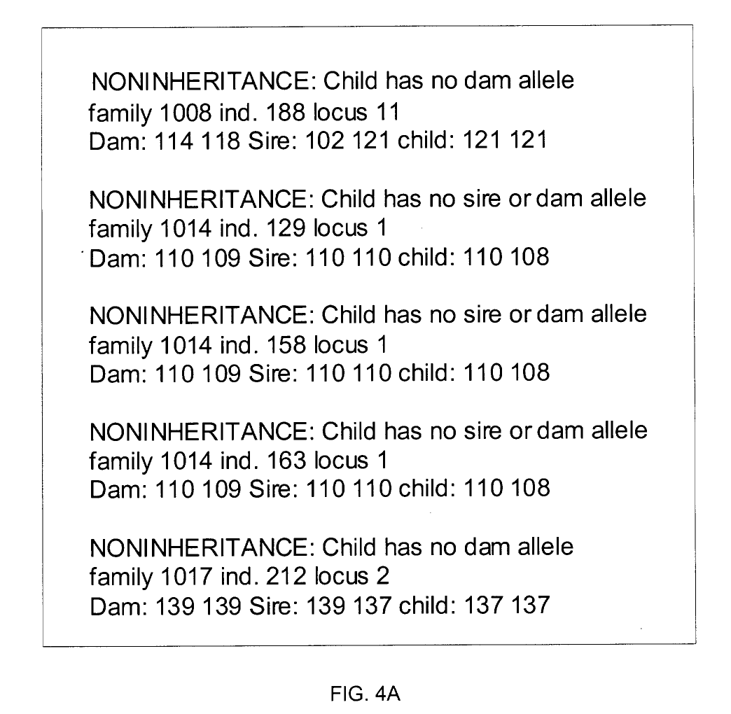

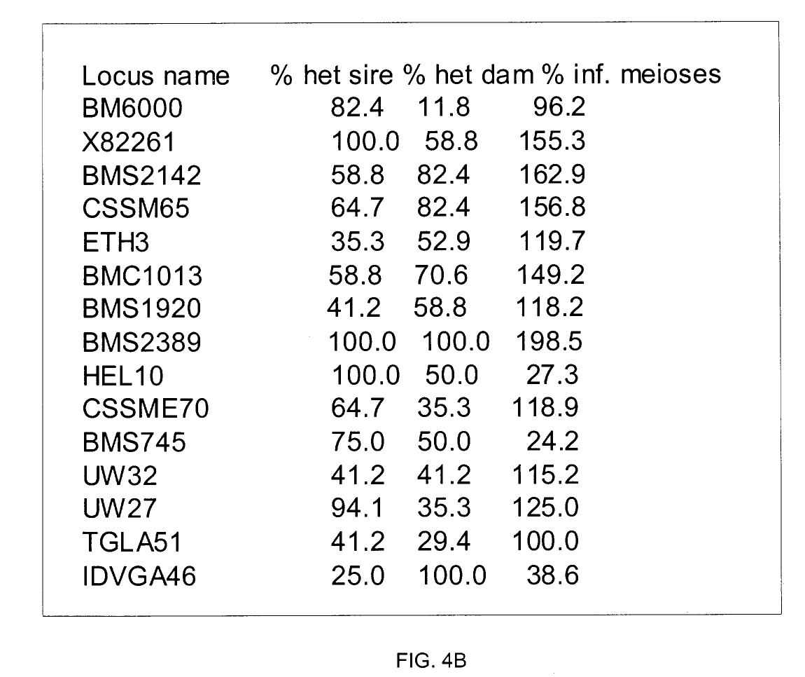

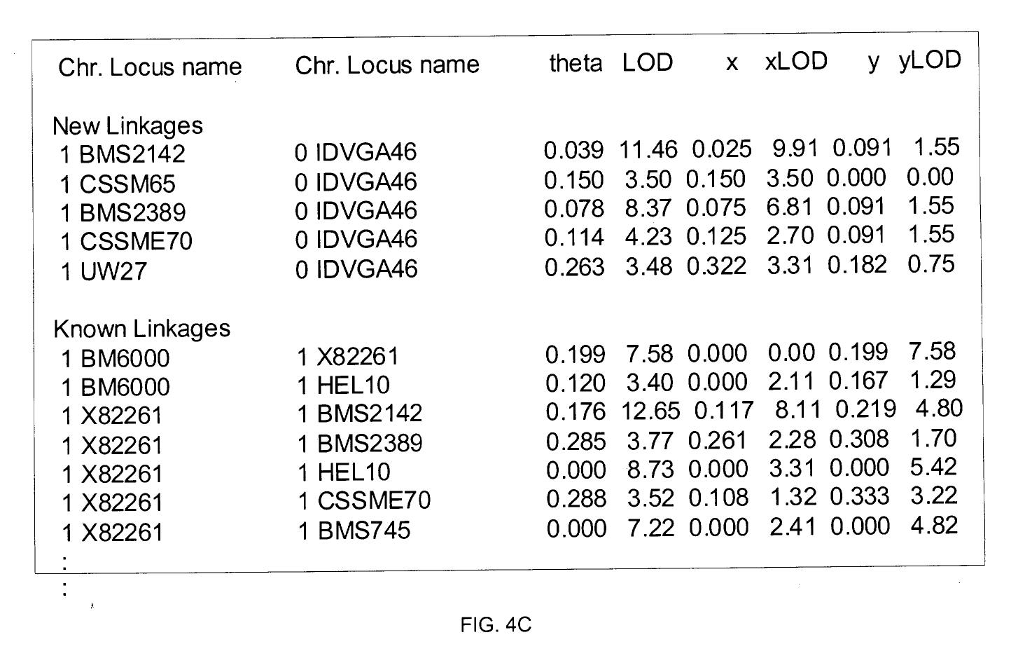

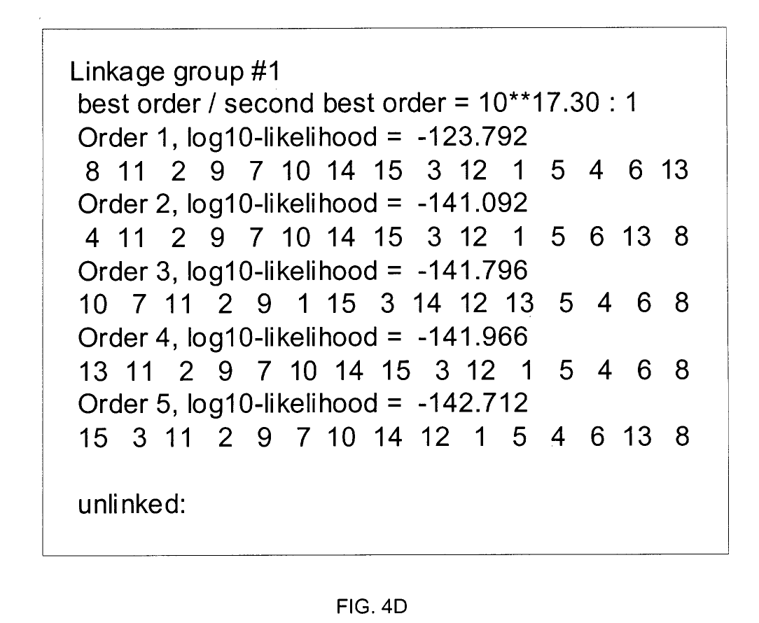

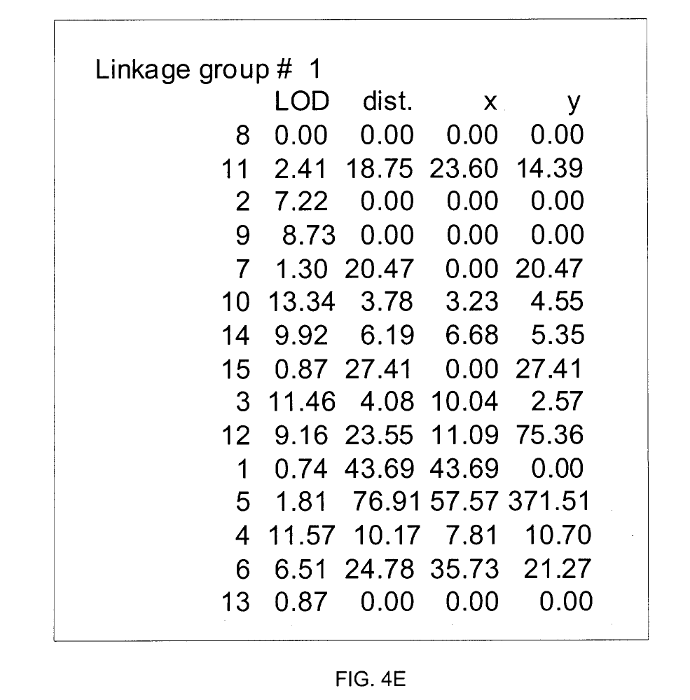

FIG. 4A is a diagram providing details of the information in data errors 112. In some embodiments of the invention, data errors file 112 include information identifying individuals where inheritance data is missing or incorrect.

FIG. 4B is a diagram providing details of the information in locus info 114. In some embodiments, locus info 114 provides information regarding a locus name and statistical information including the percentage of heterozygous sires and dames having the named locus. Additionally, a percentage of informative meioses may be provided in some embodiments. An informative meiosis has parent allele transmission. Thus the percentage of informative meioses is a rating of how informative the data is with respect to a locus. Because both a male and a female contribute to the percentage, the percentage value can range from 0 to 200%.

FIG. 4C is a diagram providing details of the information in pairwise data 116. Pairwise data file includes linkages between loci, and statistical values such as LOD scores for the linkage.

FIG. 4D is a diagram providing details of the information in locus order data 118. In some embodiments, locus order data 118 includes a series of calculated possible loci orderings, with the most likely ordering presented first in the output data stream.

FIG. 4E is a diagram providing details of the information in linkage map 120. Linkage map 120 provides statistical data regarding the individual loci for linkage groups identified during the linkage analysis.

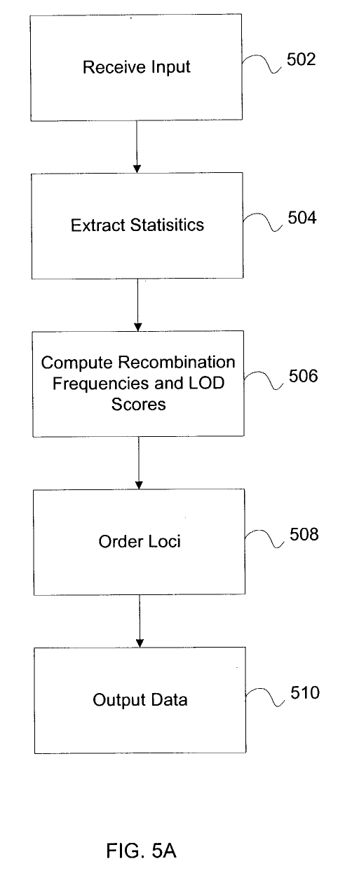

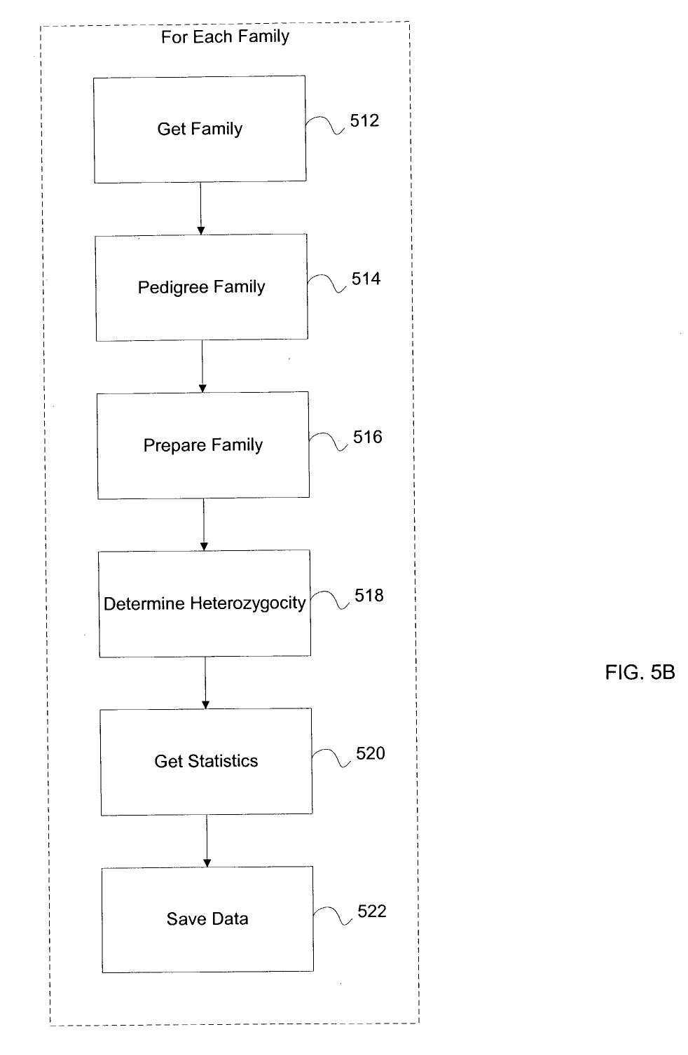

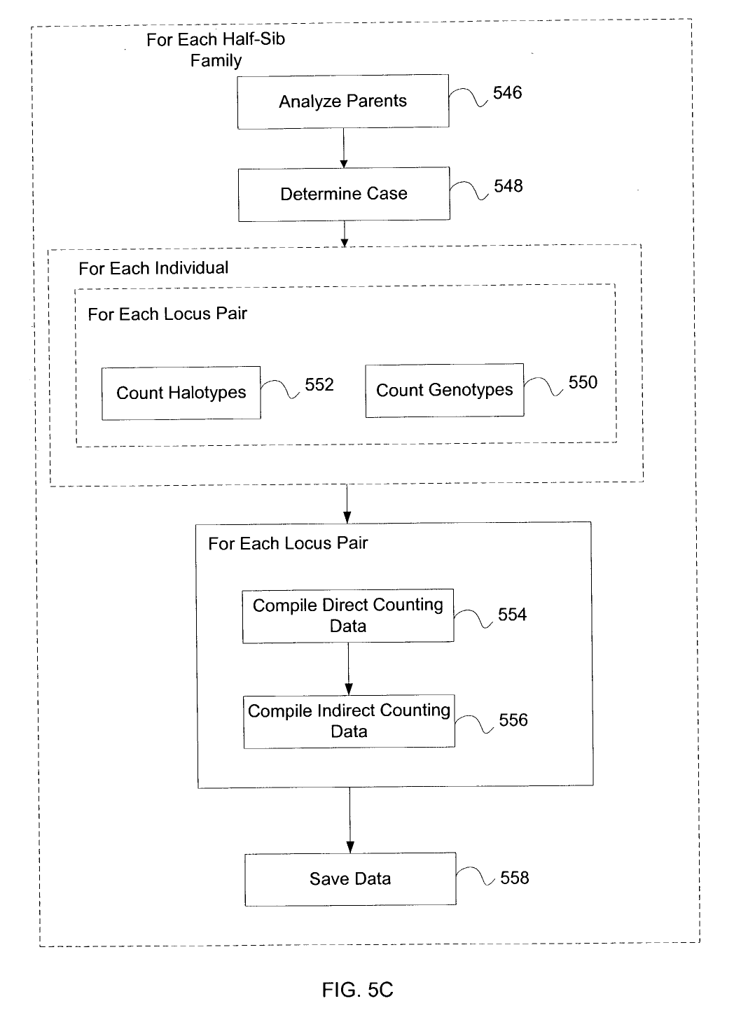

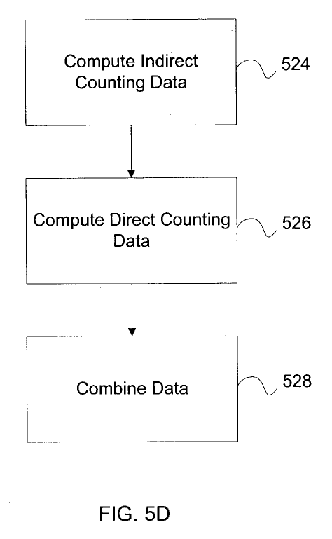

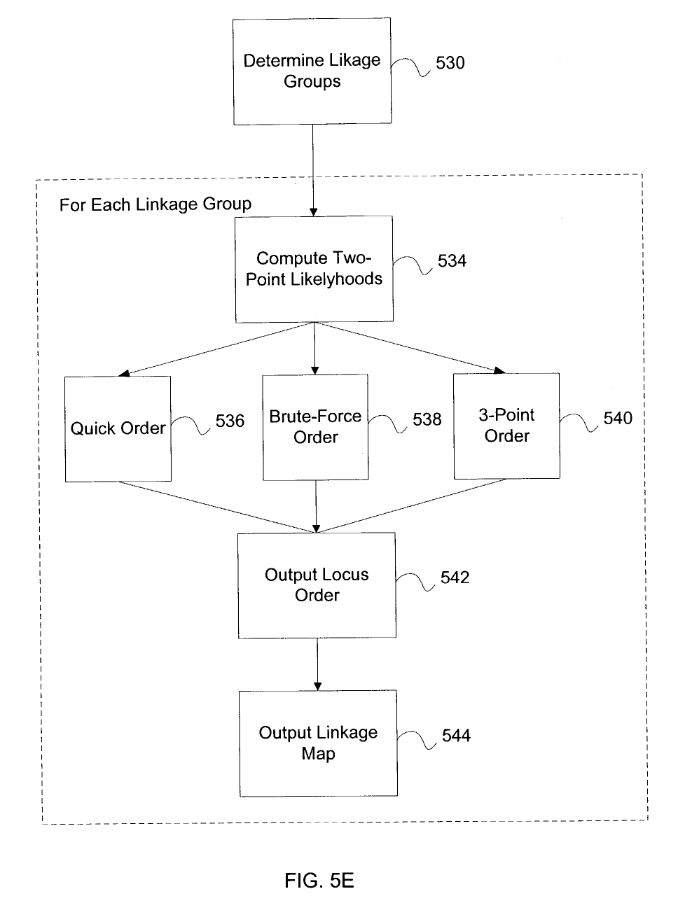

FIGS. 5A-5E are flowcharts illustrating methods for performing linkage analysis using direct and indirect counting. Direct counting is based on counting the frequencies of four haplotypes for each pair of loci and then directly computing the recombination frequency and LOD score. Indirect counting is based on counting the frequencies of genotypes for each pair of loci, and then using iterative methods to compute the recombination frequencies and LOD scores from those frequencies. The methods to be performed by the operating environment constitute one or more computer programs made up of computer-executable instructions. Describing the methods by reference to a flowchart enables one skilled in the art to develop such programs including such instructions to carry out the methods on suitable computers (the processor or processors of the computer executing the instructions from computer readable media). The methods illustrated in FIGS. 5A-5E are inclusive of acts that may be taken by an operating environment executing an exemplary embodiment of the invention.

FIG. 5A is a flowchart illustrating a method for performing linkage analysis according to some embodiments of the invention. The method begins by receiving input data (block 502). The input data typically comprises family identification data and genetic information for members of the family. Further, the input data may also include locus names data that map numeric identifiers to locus names. In addition, the input data may include parameters used to control the processing of data and for specifying the location and format for input and output data. Furthermore, control parameters may be provided on a command line for the linkage analysis program.

In some embodiments of the invention the input data may be converted from an externally defined format to an internally usable format. In some embodiments, the externally defined format is the Crimap format. In alternative embodiments, the externally defined format is the “Linkage” format.

Additionally, in some embodiments of the invention, the input data is scanned for sex-linked loci. If any such loci are found, they are flagged for special processing by later actions in the method.

Next, a system performing the method extracts statistics from input data (block 504). In some embodiments, the statistics are gathered by reading through the families in the data file one by one and counting the frequencies of haplotypes and genotypes of all locus pairs. This step essentially condenses the raw genotype and phenotype data to a condensed form that can be used for further processing.

FIG. 5B is a flowchart providing further details of the extract statistics processing of block 504. The processing illustrated in FIG. 5B will be performed for each family in the input data. Statistics extraction begins by reading data for one family from the input data (block 512). Next, a pedigree for the family is determined (block 514). The grandparents (if any), parents, and offspring are identified and ordered. Half-sib families are identified and Jo split into separate families.

Next, the family is prepared for processing (block 516). Family preparation may include all or some of the following steps:

Parents and grandparents are put in the correct order.

The data is scanned for dominant/recessive coded loci. Any such loci are checked for data errors and are prepped for processing, including converting the data to genotype data where possible.

The data is scanned for sex-influenced coded loci. Any such loci are checked for data errors and are prepped for processing, including converting the data to genotype data where possible.

The inheritance pattern of each locus is checked to make sure it is consistent across families.

The data is scanned for imprinted loci. Any such loci are checked for data errors and are prepped for processing, including converting the data to genotype data where possible.

All missing parental genotypes are filled in when they can be determined unequivocally.

Next, the heterozygocity of the family is determined (block 518). Here, the number of heterozygous parents at each locus is counted. The heterozygocity data may be used as a measure of the informativeness of a family, but is not required for indirect counting.

A system executing the method then proceeds to get statistics for the family (block 520). The statistics include genotype and haplotype frequencies that are gathered from the family data.

FIG. 5C provides further details on the get statistics processing of block 520. The system executing the methods analyzes the parent data (block 546). Here the parental alleles are ordered properly where possible and characteristics of each locus and locus pair are collected.

Next, the case of each locus pair is determined based on an inheritance mode (block 548). In some embodiments of the invention, there are thirteen cases, referred to as case 0-case 12. A case may be determined by looking at the parental alleles.

| | |
|---|---|
| Case 0: | two multiallelic, codominant loci |
| Case 1: | one biallelic codominant locus, one multiallelic |
| | codominant locus |
| Case 2: | two biallelic codominant loci |
| Case 3: | two biallelic codominant loci, mixed linkage phase |
| Case 4: | one multiallelic, codominant locus, one |
| | dominant/recessive locus |
| Case 5: | one biallelic codominant locus, one |
| | dominant/recessive locus |
| Case 6: | one biallelic codominant locus, one |
| | dominant/recessive locus, mixed |
| | linkage phase |
| Case 7: | two dominant/recessive loci, coupling phase |
| Case 8: | two dominant/recessive loci, mixed phase |
| Case 9: | two dominant/recessive loci, repulsion phase |
| Case 10: | one multiallelic codominant locus, |
| | one sex-linked locus |
| Case 11: | one biallelic codominant locus, one sex-linked locus |
| Case 12: | one biallelic codominant locus, one |
| | sex-linked locus, mixed linkage |
| | phase |

Imprinted loci are handled in a similar way as sex-linked loci. The alleles of an imprinted locus can be recoded so that the locus can be analyzed using direct counting, so imprinting does not have a case of its own.

Blocks 550 and 552 are executed for each individual in the family, and for each locus pair in the individual. Depending on the case, the haplotype frequencies are counted (block 552) and the genotype frequencies (block 550) are counted.

For each locus pair in the family, the system compiles direct counting data for locus pairs in case 0 (block 554). The haplotype frequencies are condensed into counts of recombinant and non-recombinant meioses.

In addition, the system compiles indirect counting data for each locus pair in the family that are in cases 1-12 (block 556). The list of genotype frequencies for the locus pair is sorted into proper order. If the phase can be directly determined for the locus pair, it is. Otherwise numerical methods are used to determine the phase of the locus pair. The list of genotype frequencies is reordered to compensate for the phase. The haplotype and genotype frequencies are then combined with data gathered from previous half-sib families (block 558).

Returning to FIG. 5B, after the statistics have been gathered for each half-sib family, the system saves the family data (block 522). The haplotype and genotype frequencies are combined with data gathered from previous families (full-sib).

Returning to FIG. 5A, after the statistics have been extracted for each family, the system then proceeds to compute recombination frequencies and LOD scores (block 506). The compute functions compute recombination frequencies and LOD scores for all locus pairs based on genotype frequencies and haplotype frequencies previously extracted from the raw data.

FIG. 5D is a flowchart providing further details of the compute recombination frequencies and LOD scores processing of block 506. The system computes indirect counting data for locus pairs in cases 1-12 (block 524). Using genotype frequency data determined above, recombination frequencies and LOD scores are computed for each locus pair using iterative functions. As noted above, for each locus pair, data has been gathered from several families. The same locus pair may fall into different cases in different families. For each case the recombination frequency and LOD score is computed using the appropriate functions, and then that data is combined together to give one recombination frequency and LOD score for each locus pair.

The following tables provide the formulas for computing the recombination frequency and LOD score for each case used in some embodiments of the invention. For LOD scores, an overall LOD score (Z) and a unit LOD (u) score may be provided. The unit LOD score may be defined as the expected LOD score per offspring assuming gender-average recombination frequency.

Case 1: One Biallelic Codominant Locus, One Multiallelic Codominant Locus

TABLE 1
Genotypic frequency, number of observations, and the
number of recombinants in the offspring from the
intercross of A₁B/A₂b (male) ×
A₃B/A₄b (female)

| | | | Number of | |
|---|---|---|---|---|
| | | Number | recombinants | |
| Genotype | Genotypic frequency^{a} | of observations | female^{b} | male^{b} |
| A₁A₃BB | q₁ = ¼(1 − x)(1 − y) | k₁ | 0 | 0 |
| A₁A₃bb | q₂ = ¼xy | k₂ | k₂ | k₂ |
| A₁A₄BB | q₃ = ¼x(1 − y) | k₃ | k₃ | 0 |
| A₁A₄bb | q₄ = ¼(1 − x)y | k₄ | 0 | k₄ |
| A₂A₃BB | q₅ = q₄ | k₅ | 0 | k₅ |
| A₂A₃bb | q₆ = q₃ | k₆ | k₆ | 0 |
| A₂A₄BB | q₇ = q₂ | k₇ | k₇ | k₇ |
| A₂A₄bb | q₈ = q₁ | k₈ | 0 | 0 |
| A₁A₃Bb | q₉ = q₃ + q₄ | k₉ | v₁k₉ | v₂k₉ |
| A₁A₄Bb | q₁₀ = q₁ + q₂ | k₁₀ | v₃k₁₀ | v₃k₁₀ |
| A₂A₃Bb | q₁₁ = q₁ + q₂ | k₁₁ | v₃k₁₁ | v₃k₁₁ |
| A₂A₄Bb | q₁₂ = q₃ + q₄ | k₁₂ | v₁k₁₂ | v₂k₁₂ |
| Total | 1 | n | nₓ | n_{y} |

^{a}x = female recombination frequency, y = male recombination frequency.
^{b}v₁ = x(1 − y)/[x(1 − y) + (1− x)y], v₂ = (1 − x)y/[x(1 − y) + (1 − x)y], v₃ = xy/[xy + (1 − x)(1 − y)].

From Table 1, gender-specific recombination frequencies may be obtained by the following iterative solutions:

$$\begin{matrix}\begin{matrix}{x}^{( i + 1 )} = a + \frac{{bx}^{( i )} ( 1 - {y}^{( i )} )}{{x}^{( i )} ( 1 - {y}^{( i )} ) + ( 1 - {x}^{( i )} ) {y}^{( i )}} + \\ \frac{{cx}^{( i )} {y}^{( i )}}{( 1 - {x}^{( i )} ) ( 1 - {y}^{( i )} ) + {x}^{( i )} {y}^{( i )}}\end{matrix} & ( 1 ) \\ \begin{matrix}{y}^{( i + 1 )} = d + \frac{b ( 1 - {x}^{( i )} ) {y}^{( i )}}{{x}^{( i )} ( 1 - {y}^{( i )} ) + ( 1 - {x}^{( i )} ) {y}^{( i )}} + \\ \frac{{cx}^{( i )} {y}^{( i )}}{( 1 - {x}^{( i )} ) ( 1 - {y}^{( i )} ) + {x}^{( i )} {y}^{( i )}}\end{matrix} & ( 2 )\end{matrix}$$

where x=female recombination frequency, y=male recombination frequency, superscript i=iteration number, a=(k₂+k₃+k₆+k₇)/n, b=(k₉+k₁₂)/n, c=(k₁₀+k₁₁)/n, and d=(k₂+k₄+k₅+k₇)/n, and where k₁ through k₁₂ are defined in Table 1. The gender-average recombination frequency can be estimated as θ=(x+y)/2, noting that the male and female parents have the same number of meioses. This method of estimating gender-average recombination frequency may also be used for other cases where gender-specific recombination frequencies are available.

LOD scores may be determined according to the following:

Zₓ=N₁log₁₀[2(1−x)]+N₂log₁₀(2x)+N₃log₁₀[2x(1−y)+2(1−x)y]+N₄log₁₀[2xy+2(1−x)(1−y)] (3)

Z_{y}=N₅log₁₀[2(1−y)]+N₆log₁₀(2y)+N₃log₁₀[2x(1−y)+2(1−x)y]+N₄log₁₀[2xy+2(1−x)(1−y)] (4)

Z_{θ}=N₇log₁₀[4(1−θ)²]+N₈log₁₀(4θ²)+N₉log₁₀[4θ(1−θ)]+N₁₀log₁₀{2[(1−θ)²+θ²]} (5)

u=½(1−θ)²log₁₀[4(1−θ)²]+½θ²log₁₀(4θ²)+2θ(1−θ)log₁₀[4θ(1−θ)]

+½[(1−θ)²+θ²]log₁₀{2[(1−θ)²+θ²]} (6)

where N₁=k₁+k₄+k₅+k₈, N₂=k₂+k₃+k₆+k₇, N₃=k₉+k₁₂, N₄=k₁₀+k₁₁, N₅=k₁+k₃+k₆+k₈, N₆=k₂+k₄+k₅+k₇, N₇=k₁+k₈, N₈=k₂+k₇, N₉=k₃+k₄+k₅+k₆+k₉+k₁₂, and N₁₀=k₁₀+k₁₁.

Case 2: Two Biallelic Codominant Loci

TABLE 2
Genotypic frequency, number of observations, and
the number of recombinants in the offspring
from the intercross of AB/ab × AB/ab

| | | Number of | Number of |
|---|---|---|---|
| Genotype | Genotypic frequency^{a} | observations | recombinants |
| AABB | q₁ = ¼(1 − θ)² | k₁ | 0 |
| AABb | q₂ = ½θ(1 − θ) | k₂ | k₂ |
| AAbb | q₃ = ¼θ² | k₃ | 2k₃ |
| AaBB | q₄ = q₂ | k₄ | k₄ |
| AaBb | q₅ = 2(q₁ + q₃) | k₅ | 2k₅θ²/[(1 − θ)² + θ²] |
| Aabb | q₆ = q₂ | k₆ | k₆ |
| aaBB | q₇ = q₃ | k₇ | 2k₇ |
| aaBb | q₈ = q₂ | k₈ | k₈ |
| aabb | q₉ = q₁ | k₉ | 0 |
| Total | 1 | n | nᵣ |

^{a}θ = gender-average recombination frequency.

For this case, gender-specific recombination frequencies are unavailable and gender-average recombination frequency can be estimated based on Table 2. The resulting formula is:

$$θ = {[ - s + \sqrt{{s}^{2} + {t}^{3}} ]}^{\frac{1}{3}} - {[ s + \sqrt{{s}^{2} + {t}^{3}} ]}^{\frac{1}{3}} + \frac{{a}_{1}}{3} \qquad ( 7 )$$

where s=½[a₁a₂/3−(2/27)a₁³−c], t=⅓(a₂−a₁²/3), a₁=(T+c₁+n₄)/T, a₂=0.5+c₁/T, and where T=2n, c₁=2n₃+n₂, c=c₁/(2T), n₁=k₁+k₉, n₂=k₂+k₄+k₆+k₈, n₃=k₃+k₇, and n₄=k₅. Note that equation (7) is derived under the assumption of coupling parental linkage phases but is applicable to the repulsion linkage phases by reversing the allele definitions for one of the two loci.

LOD scores may be determined according to the following:

Z_{θ}=N₁log₁₀[4(1−θ)²]+N₂log₁₀[4θ(1−θ)]+N₃log₁₀(4θ²)+N₄log₁₀{2[(1−θ)²+θ²]} (8)

where N₁=k₁+k₉, N₂=k₂+k₄+k₆+k₈, N₃=k₃+k₇, N₄=k₅. The unit LOD score is the same as that for the MB data type.

Case 3: Two Biallelic Codominant Loci, Mixed Linkage Phase

TABLE 3
Offspring phenotypes and recombinants from
the mating of AB/ab (male) × Ab/aB (female)

| | | | Number of | |
|---|---|---|---|---|
| | | Number | recombinants | |
| Genotype | Genotypic frequency | of observations | female^{a} | male^{a} |
| AABB | q₁ = ¼x(1 − y) | k₁ | k₁ | 0 |
| AABb | q₂ = ¼[(1 − x)(1 − y) + xy] | k₂ | v₁k₂ | v₁k₂ |
| AAbb | q₃ = ¼(1 − x)y | k₃ | 0 | k₃ |
| AaBB | q₄ = q₂ | k₄ | v₁k₄ | v₁k₄ |
| AaBb | q₅ = ½[x(1 − y) + (1 − x)y] | k₅ | v₃k₅ | v₂k₅ |
| Aabb | q₆ = q₂ | k₆ | v₁k₆ | v₁k₆ |
| aaBB | q₇ = q₃ | k₇ | 0 | k₇ |
| aaBb | q₈ = q₂ | k₈ | v₁k₈ | v₁k₈ |
| aabb | q₉ = q₁ | k₉ | k₉ | 0 |
| Total | 1 | n | nₓ | n_{y} |

^{a}v₁ = xy/[(1 − x)(1 − y) + xy] v₂ = (1 − x)y/[x(1 − y) + (1 − x)y] v₃ = x(1 − y)/[x(1 − y) + (1 − x)y]

From Table 3, gender-specific recombination frequencies can be obtained by the following iterative solutions:

x⁽ⁱ⁺¹⁾=a+[bx⁽ⁱ⁾(1−y⁽ⁱ⁾)]/[x⁽ⁱ⁾(1−y⁽ⁱ⁾)+(1−x⁽ⁱ⁾)y⁽ⁱ⁾]+cx⁽ⁱ⁾uy⁽ⁱ⁾/[(1−x⁽ⁱ⁾)(1−y⁽ⁱ⁾)+x⁽ⁱ⁾y⁽ⁱ⁾] (9)

y⁽ⁱ⁺¹⁾=d+[b(1−x⁽ⁱ⁾)y⁽ⁱ⁾]/[x⁽ⁱ⁾(1−y⁽ⁱ⁾)+(1−x⁽ⁱ⁾)y⁽ⁱ⁾]+cx⁽ⁱ⁾y⁽ⁱ⁾/[(1−x⁽ⁱ⁾)(1−y⁽ⁱ⁾)+x⁽ⁱ⁾y⁽ⁱ⁾] (10)

where x=female recombination frequency, y=male recombination frequency, a=(k₁+k₉)/n, b=k₅/n, c=(k₂+k₄+k₆+k₈)/n, and d=(k₃+k₇)/n.

LOD scores may be determined according to the following:

Zₓ=(k₁+k₉)log₁₀(2x)+(k₂+k₄+k₆+k₈)log₁₀{2[(1−x)(1−y)+xy)]}+(k₃+k₇)log₁₀[2(1−x)]+k₅log₁₀{2[x(1−y)+y(1−x)]} (11)

Z_{y}=(k₁+k₉)log₁₀[2(1−y)]+(k₂+k₄+k₆+k₈)log₁₀{2[(1−x)(1−y)+xy)]}+(k₃+k₇)log₁₀(2y)+k₅log₁₀{2[x(1−y)+y(1−x)]} (12)

Z_{θ}=(k₁+k₃+k₅+k₇+k₉)log₁₀[4θ(1−θ)]+(k₂+k₄+k₆+k₈)log₁₀{2[(1−θ)²+θ²)]} (13)

u=2θ(1−θ) log[4θ(1−θ)]+(1−2θ+2θ²)log[2(1−2θ+2θ²)] (14)

Case 4: One Multiallelic, Codominant Locus, One Dominant/Recessive Locus

TABLE 4
Genotypic frequency, number of observations, and the
number of recombinants in the offspring from the
intercross of A₁B/A₂b (male) ×
A₃B/A₄b (female) with B being
dominant over b

| | Genotypic | Number of | Number of recombinants | |
|---|---|---|---|---|
| Genotype | frequency | observations | female | male |
| A₁A₃bb | q₁ = ¼xy | k₁ | k₁ | k₁ |
| A₁A₄bb | q₂ = ¼(1 − x)y | k₂ | 0 | k₂ |
| A₂A₃bb | q₃ = ¼x(1 − y) | k₃ | k₃ | 0 |
| A₂A₄bb | q₄ = ¼(1 − x)(1 − y) | k₄ | 0 | 0 |
| A₁A₃B- | q₅ = ¼(1 − xy) | k₅ | k₅x(1 − y)/(1 − xy) | k₅(1 − x)y/(1 − xy) |
| A₁A₄B- | q₆ = ¼[1 − (1 − x)y] | k₆ | k₆x/[1 − (1 − x)y] | k₆xy/[1 − (1 − x)y] |
| A₂A₃B- | q₇ = ¼[1 − x(1 − y)] | k₇ | k₇xy/[1 − x(1 − y)] | k₇y/[1 − x(1 − y)] |
| A₂A₄B- | q₈ = ¼(x + y − xy) | k₈ | k₈x/(x + y − xy) | k₈y/(y + x − xy) |
| Total | 1 | n | nₓ | n_{y} |

From Table 4, gender-specific recombination frequencies can be obtained by the following iterative solutions:

$$\begin{matrix}{x}^{( i + 1 )} = \frac{{ax}^{( i )} ( 1 - {y}^{( i )} )}{1 - {x}^{( i )} {y}^{( i )}} + \frac{{bx}^{( i )}}{( 1 - {x}^{( i )} ) {y}^{( i )}} + \frac{{cx}^{( i )} {y}^{( i )}}{1 - {x}^{( i )} ( 1 - {y}^{( i )} )} + \frac{{dx}^{( i )}}{{x}^{( i )} + {y}^{( i )} - {x}^{( i )} {y}^{( i )}} + e & ( 15 ) \\ {y}^{( i + 1 )} = \frac{a ( 1 - {x}^{( i )} ) {y}^{( i )}}{1 - {x}^{( i )} {y}^{( i )}} + \frac{{bx}^{( i )} {y}^{( i )}}{( 1 - {x}^{( i )} ) {y}^{( i )}} + \frac{{cy}^{( i )}}{1 - {x}^{( i )} ( 1 - {y}^{( i )} )} + \frac{{dy}^{( i )}}{{x}^{( i )} + {y}^{( i )} - {x}^{( i )} {y}^{( i )}} + f & ( 16 )\end{matrix}$$

where a=k₅/n, b=k₆/n, c=k₇/n, d=k₉/n, e=(k₁+k₂)/n, and f=(k₁+k₃)/n.

LOD scores may be determined according to the following:

Z_{z}=(k₁+k₃)log₁₀(2x)+(k₂+k₄)log₁₀[2(1−x)]+k₅log₁₀[[2(1−xy)/(2−y)]

+k₆log₁₀{2[1−(1−x)y]/(2−y)}+k₇log₁₀{2[(1−x(1−y)]/(1+y)}+k₉log₁₀[2(x+y−xy)/(1+y)] (17)

Z_{y}=(k₁+k₂)log₁₀(2y)+(k₃+k₄)log₁₀[2(1−y)]+k₅log₁₀[[2(1−xy)/(2−x)]

+k₆log₁₀{2[(1−x(1−y)]/(1+x)}+k₇log₁₀{2[1−(1−x)y]/(2−y)}+k₈log₁₀[2(x+y−xy)/(1+y)] (18)

Z_{θ}=k₁log₁₀(4θ²)+(k₂+k₃)log₁₀[4θ(1−θ)]+k₄log₁₀[4(1−θ)²]+k₅log₁₀[(4/3)(1−θ²)]

+(k₆+k₇)log₁₀{(4/3)[1−θ(1−θ)]}+k₈log₁₀[(4/3)θ(2−θ)] (19)

u=¼θ²logg₁₀(4θ²)+½θ(1−θ)log₁₀[4θ(1−θ)]+¼(1−θ)²log₁₀[4(1−θ)²]

+1/4(1−θ²)log₁₀[(4/3)(1−θ²)]+1/2[(1−θ(1−θ)]log₁₀{(4/3)[1−θ(1−θ)]}+¼θ(2−θ)log₁₀[(4/3)θ(2−θ)] (20)

Case 5: One Biallelic Codominant Locus, One Dominant/Recessive Locus

TABLE 5
Genotypic frequency, number of observations, and
the number of recombinants in the offspring from
the intercross of AB/ab × AB/ab with
allele B being dominant over allele b

| | Genotypic | Number of | Number of |
|---|---|---|---|
| Genotype | frequency | observations | recombinants |
| AAB- | q₁ = ¼(1 − θ) | k₁ | 2k₁θ/(1 + θ) |
| | (1 + θ) | | |
| AAbb | q₂ = ¼θ² | k₂ | 2k₂ |
| AaB- | q₃ =½[1 − θ(1 − θ)] | k₃ | k₃θ(1 + θ)/[1 − θ(1 − θ)] |
| Aabb | q₄ = ½θ(1 − θ) | k₄ | k₄ |
| aaB- | q₅ = ¼θ(2 − θ) | k₅ | 2k₅/(2 − θ) |
| aabb | q₆ = ¼(1 − θ)² | k₆ | 0 |
| Total | 1 | n | nᵣ |

Gender-specific recombination frequencies are generally nonestimable for this case. From Table 5, the gender-average recombination frequency may be obtained using the following iterative solution:

θ⁽ⁱ⁺¹⁾=a+bθ⁽ⁱ⁾/(1+θ⁽ⁱ⁾)+cθ⁽ⁱ⁾(1+θ⁽ⁱ⁾)/[1−θ⁽ⁱ⁾(1−θ^{(i))]+}d/(2−θ⁽ⁱ⁾) (21)

where a=(2k₂+k₄)/(2n), b=k₁/n, c=k₃/(2n), and d=k₅/n.

LOD scores may be determined according to the following:

Z_{θ}=k₁log₁₀[(4/3)(1−σ²)]+k₂log₁₀(4θ²)+k₃log₁₀{(4/3)[1−θ(1−θ)]}+k₄log₁₀[4θ(1−θ)]

+k₅log₁₀{(4/3)θ(2−θ)]}+k₆log₁₀[4(1−θ)²] (22)

The unit LOD score is the same as equation 20 above.

Case 6: One Biallelic Codominant Locus, One Dominant/Recessive Locus, Mixed Linkage Phase

TABLE 6
Offspring phenotypes and recombinants from the mating
of AB/ab (male) × Ab/aB (female)

| | | | Number of | |
|---|---|---|---|---|
| | | Number | recombinants | |
| Genotype | Genotypic frequency | of observations | female^{a} | male^{a} |
| AAB_{—} | q₁ = ¼(1 − y + xy) | k₁ | k₁v₁ | k₁v₂ |
| AAbb | q₂ = ¼(1 − x)y | k₂ | 0 | k₂ |
| AaB_{—} | q₃ = ¼(1 + x + y − 2xy) | k₃ | k₃v₃ | k₃v₄ |
| Aabb | q₄ = ¼((1 − x)(1 − y) + xy) | k₄ | k₄v₅ | k₄v₆ |
| aaB_{—} | q₅ = ¼(1 − x + xy) | k₅ | k₅v₇ | k₅v₈ |
| aabb | q₆ = ¼x(1 − y) | k₆ | k₆ | 0 |
| Total | 1 | n | nₓ | n_{y} |
| ^{a}v₁ = [x(1 − y) + xy]/(1 − y + xy), v₂ = xy/(1 − y + xy), v₃ = 2[x(1 − y) + xy]/(1 + x + y − 2xy), v₄ = 2[(1 − x)y + xy]/(1 + x + y − 2xy), v₅ = xy/[(1 − x)(1 − y) + xy], v6 = [x + (1 − x)y]/[(1 − x) | | | | |
| # (1 − y) + xy], v₇ = xy/(1 − x + xy), v₈ = [(1 − x) | | | | |

From Table 6, gender-specific recombination frequencies may be obtained by the following iterative solutions:

x⁽ⁱ⁺¹⁾=av₁⁽ⁱ⁾+cv₃⁽ⁱ⁾+dv₅⁽ⁱ⁾+ev₇⁽ⁱ⁾+f (23)

y⁽ⁱ⁺¹⁾=av₂⁽ⁱ⁾+b+cv₄⁽ⁱ⁾+dv₆⁽ⁱ⁾+ev₈⁽ⁱ⁾ (24)

where a=k₁/n, b=k₂/n, c=k₃/n, d=k₄/n, e=k₅/n, f=k₆/n, ^{a}v₁=[x(1−y)+xy]/(1−y+xy), v₂=xy/(1−y+xy), v₃=2[x(1−y)+xy]/(1+x+y−2xy), v₄=2[(1−x)y+xy]/(1+x+y−2xy), v₅=xy/[(1−x)(1−y)+xy], v₆=[x+(1−x)y]/[(1−x)(1−y)+xy], v₇=xy/(1−x+xy), v₈=[(1−x)y+xy]/(1−x+xy).

LOD scores may be determined according to the following:

Zₓ=k₁log₁₀[2(1−y+xy)/(2−y)]+k₂log₁₀[2(1−x)]+k₃log₁₀[(2/3)(1+x+y−2xy)]

+k₄log₁₀{2[(1−x)(1−y)+xy)]}+k₅log₁₀[2(1−x+xy)/(1+y)]+k₆log₁₀(2x) (25)

Z_{y}=k₁log₁₀[2(1−y+xy)/(1+x)]+k₂log₁₀(2y)+k₃log₁₀[(2/3)(1+x+y−2xy)]

+k₄log₁₀{2[(1−x)(1−y)+xy)]}+k₅log₁₀[2(1−x+xy)/(2−x)]+k₆log₁₀[2(1−y)] (26)

Z_{θ}=(k₁+k₅)log₁₀[(4/3)(1−θ+θ²)]+(k₂+k₆)log₁₀[4θ(1−θ)]+k₃log₁₀[(2/3)(1+2θ−2θ²)]

+k₄log₁₀{2[(1−θ)2+θ²)]} (27)

u=[½(1−θ+θ²)]log₁₀[(4/3)(1−θ+θ²)]+[½θ(1−θ)]log₁₀[4θ(1−θ)]

+[¼(1+2−2θ²)]log₁₀[(2/3)(1+2θ−2θ²)]+{[(1−θ)2+θ²)]}log₁₀{2[(1−θ)2+θ²)]} (28)

Case 7: Two Dominant/Recessive Loci, Coupling Phase

TABLE 7
Genotypic frequency, number of observations, and the
number of recombinants in the offspring from the
intercross of AB/ab × AB/ab with allele A being
dominant over a and B being dominant over b

| | Genotypic | Number of | |
|---|---|---|---|
| Genotype | frequency^{a} | observations | Number of recombinants |
| A-B- | q₁ = ¼[2 + (1 − θ)²] | k₁ | 4k₁θ(1 + θ)/[2 + |
| | | | (1 − θ)²] |
| A-bb | q₂ = ¼θ(2 − θ) | k₂ | 2k₂/(2 − θ) |
| aaB- | q₃ = ¼θ(2 − θ) | k₃ | 2k₃/(2 − θ) |
| aabb | q₄ = ¼(1 − θ)² | k₄ | 0 |
| Total | 1 | n | nᵣ |

In this case, both parents are assumed to have coupling linkage phase (Table 7). The gender-average recombination frequency can be obtained from the following iterative solution:

θ⁽ⁱ⁺¹⁾=4aθ⁽ⁱ⁾(1+θ⁽ⁱ⁾)/[2+(1−θ⁽ⁱ⁾)²]+2b/(2−θ⁽ⁱ⁾) (29)

where a=k₁/(2n), and b=(k₂+k₃)/(2n).

LOD scores may be determined according to the following:

Z₀=k₁log₁₀{(8/9)[1+0.5(1−θ)²]}+(k₂+k₃)log₁₀[(4/3)θ(2−θ)]+k₄log₁₀[4(1−θ)²] (30)

u=q₁log₁₀{(8/9)[1+0.5(1−θ)²]}+(q₂+q₃)log₁₀[(4/3)θ(2−θ)]+q₄log₁₀[4(1−θ)²] (31)

Case 8: Two Dominant/Recessive Loci, Mixed Phase

TABLE 8
Genotypic frequency, number of observations, and the
number of recombinants in the offspring from the
intercross of AB/ab × Ab/aB with allele A being
dominant over a and B being dominant over b

| Gen- | | Number of | |
|---|---|---|---|
| otype | Genotypic frequency^{a} | observations | Number of recombinants |
| A-B- | q₁ = ¼[2 + θ(1 − θ)] | k₁ | k₁θ(5 − θ)/[2 + θ(1 − θ)] |
| A-bb | q₂ = ¼[1 − θ(1 − θ)] | k₂ | k₂θ(1 + θ)/[1 − θ(1 − θ)] |
| aaB- | q₃ = ¼[1 − θ(1 − θ)] | k₃ | k₃θ(1 + θ)/[1 − θ(1 − θ)] |
| aabb | q₄ = ¼θ(1 − θ) | k₄ | k₄ |
| Total | 1 | n | nᵣ |

In this case, one parent is assumed to have coupling phase and the other repulsion phase. The gender-average recombination frequency can be obtained from the following iterative solution:

θ⁽ⁱ⁺¹⁾=aθ⁽ⁱ⁾(5−θ⁽ⁱ⁾)/[2+θ⁽ⁱ⁾(1−θ⁽ⁱ⁾)]+bθ⁽ⁱ⁾(1+θ⁽ⁱ⁾)/[1−θ⁽ⁱ⁾(1−θ⁽ⁱ⁾)]+c (32)

where a=k₁/(2n), b=(k₂+k₃)/(2n), and c=k₄/(2n). For the case when the two loci are dominant and both parents have repulsion linkage phase (DD-RR data type), an analytical formula for maximum likelihood estimation of recombination frequency may be used.

LOD scores may be determined according to the following:

Z_{θ}=k₁log₁₀{(8/9)[1+½θ(1−θ)]}+(k₂+k₃)log₁₀{(4/3)[1−θ(1−θ)]}+k₄log₁₀[4θ(1−θ)] (33)

u=q₁log₁₀{(8/9)[1+½θ(1−θ)]}+(q₂+q₃)log₁₀{(4/3)[1−θ(1−θ)]}+q₄log₀[4θ(1−θ)] (34)

Case 9: Two Dominant/Recessive Loci, Repulsion Phase

TABLE 9
Genotypic frequency, number of observations, and the
number of recombinants in the offspring from the
intercross of Ab/aB Ab/aB with allele A being dominant
over a and B being dominant over b.

| | | Number of | |
|---|---|---|---|
| Genotype | Genotypic frequency | Observations | Expected recombinants |
| A_B_{—} | p₁ = ¼(2 + θ²) | k₁ | k₁ θ(2 + θ)/(2 + θ²) |
| A_bb | p₂ = ¼(1 − θ²) | k₂ | k₂ θ/(1 + θ) |
| aaB_{—} | p₃ = ¼(1 − θ²) | k₃ | k₃ θ/(1 + θ) |
| aabb | p₄ = ¼θ² | k₄ | k₄ |
| Total | 1 | n | nᵣ |

The recombination frequency may be obtained from the following:

θ={[−(2k1−4(k2+k3)−2k4)±√{[2k1−4(k2+k3)−2k4]²+8[2(k1+k2+k3)+2k4]2k4}]/[−2[2(k1+k2+k3)+2k4]]}² (35)

LOD scores may be determined according to the following:

Zₓ=nlog₁₀(2)+(k₂+k₄)log₁₀(x)+(k₁+k₃)log₁₀(1−x) (36)

Z_{y}=nlog₁₀(2)+(k₃+k₄)log₁₀(y)+(k₁+k₂)log₁₀(1−y) (37)

Z_{θ}=2nlog₁₀(2)+(k₂+k₃+2k₄)log₁₀(θ)+(2k₁+k₂+k₃)log₁₀(1−θ) (38)

u=2[log₁₀(2)+θlog₁₀(θ)+(1−θ)log₁₀(1−θ)] (39)

Case 10: One Multiallelic Codominant Locus, One Sex-Linked Locus

TABLE 10
Offspring phenotypes and recombinants from the mating of A₁B/A₂b × A₃B/A₄b.

| Genotype and | | Number of | | | | Male | | Female | |
|---|---|---|---|---|---|---|---|---|---|
| phenotype | | offspring | | Frequency | | recombinants | | recombinants | |
| Marker | Trait | M | F | M | F | M^{a} | F | M^{a} | F |
| A₁A₃ | expressed | m₁ | f₁ | p₁ = ¼(1 − xy) | p₈ | q₁ = ¼y(1 − x)/p₁ | 0 | q₅ = ¼x(1 − y)/p₁ | 0 |
| A₁A₄ | expressed | m₂ | f₂ | p₂ = ¼(1 − y + xy) | p₇ | q₂ = ¼xy/p₂ | 0 | q₆ = ¼β/p₂ | 1 |
| A₂A₃ | expressed | m₃ | f₃ | p₃ = ¼(1 − x + xy) | p₆ | q₃ = ¼α/p₃ | 1 | q₇ = ¼xy/p₃ | 0 |
| A₂A₄ | expressed | m₄ | f₄ | p₄ = ¼(x + y − xy) | p₅ | q₄ = ¼α/p₄ | 1 | q₈ = ¼β/p₄ | 1 |
| A₁A₃ | unexpressed | m₅ | f₅ | p₅ = ¼xy | p₄ | 1 | q₄ | 1 | q₈ |
| A₁A₄ | unexpressed | m₆ | f₆ | p₆ = ¼(1 − x)y | p₃ | 1 | q₃ | 0 | q₇ |
| A₂A₃ | unexpressed | m₇ | f₇ | p₇ = ¼x(1 − y) | p₂ | 0 | q₂ | 1 | q₆ |
| A₂A₄ | unexpressed | m₈ | f₈ | p₈ = ¼(1 − x)(1 − y) | p₁ | 0 | q₁ | 0 | q₅ |

^{a}α = y(1 − x) + xy, β = x(1 − y) + xy

The recombination frequency may be obtained from the following:

θ⁽ⁱ⁺¹⁾=aλ₃⁽ⁱ⁾+bλ₂⁽ⁱ⁾+2cλ₁⁽ⁱ⁾+2g+e for Ab/aB×Ab/aB (40)

where λ₁=θ/(1+θ), λ₂=θ(1+θ)/(1−θ+θ²), λ₃=1/(1−½θ), λ₄=θ/(1+2θ−2θ²), λ₅=θ/(1−2θ+2θ²), a=(m₁+f₆)/2n, b=(m₂+f₅)/2n, c=(m₃+f₄)/2n, d=(m₄+f₃)/2n, e=(m₅+f₂)/2n, g=(m₆+f₁)/2n, and where m₁ and f₁ are defined in Table 10.

LOD scores may be determined according to the following:

U_{F}=¼(1−θ²)log[4(1−θ²)/3]+½(1−θ+θ²)log[4(1−θ+θ²)/3]+¼θ(2−θ)log[4θ(2−θ)/3]

+1/4θ²log(4θ²)+½θ(1−θ)log[4θ(1−θ)]+¼(1−θ)²log[4(1−θ)²] (41)

Case 11: One Biallelic Codominant Locus, One Sex-Linked Locus

TABLE 11
Offspring phenotypes and recombinants from the mating of AB/ab × AB/ab.

| | | | |
|---|---|---|---|
| Genotype and | Number of | | Observed and expected |
| phenotype | offspring | Frequency | recombinants |

| Marker | Trait | M^{a} | F^{a} | M^{a} | F^{a} | M^{a} | F^{a} |
|---|---|---|---|---|---|---|---|
| AA | expressed | m₁ | f₁ | p₁ = ¼(1 − θ²) | p₆ | ½θ(1 − θ)/p₁ | 0 |
| Aa | expressed | m₂ | f₂ | p₂ = ½(1 − θ + θ²) | p₅ | ½θ(1 + θ)/p₂ | 1 |
| aa | expressed | m₃ | f₃ | p₃ = ¼θ(2 − θ) | p₄ | ½θ/p₃ | 2 |
| AA | unexpressed | m₄ | f₄ | p₄ = ¼θ² | p₃ | 2 | ½θ/p₄ |
| Aa | unexpressed | m₅ | f₅ | p₅ = ½θ(1 − θ) | p₂ | 1 | ½θ(1 + θ)/p₅ |
| aa | unexpressed | m₆ | f₆ | p₆ = ¼(1 − θ)² | p₁ | 0 | ½θ(1 − θ)/p₆ |

^{a}M and F denote the male and female offspring respectively

The recombination frequency may be obtained from the following:

θ⁽ⁱ⁺¹⁾=2aλ₁⁽ⁱ⁾+bλ₂⁽ⁱ⁾+cλ₃⁽ⁱ⁾+2d+e (42)

where λ₁=θ/(1+θ), λ₂=θ(1+θ)/(1−θ+θ²), λ₃=1/(1−½θ), λ₄=θ/(1+2θ−2θ²), λ_{5=θ/(}1−2θ+2θ²), a=(m₁+f₆)/2n, b=(m₂+f₅)/2n, c=(m₃+f₄)/2n, d=(m₄+f₃)/2n, e=(m₅+f₂)/2n, g=(m₆+f₁)/2n, and where mᵢ and fᵢ are defined in Table 11.

LOD scores may be determined according to formula 41 above.

Case 12: One Biallelic Codominant Locus, One Sex-Linked Locus, Mixed Linkage Phase

TABLE 12
Offspring phenotypes and recombinants from the mating of AB/ab × aB/Ab.

| Genotype/ | | | | | | | |
|---|---|---|---|---|---|---|---|
| Phenotype | | Number Frequency | | | | Recombinants | |
| Mark | Trait | M^{a} | F^{a} | M^{a} | F^{a} | M^{a} | F^{a} |
| AA | expressed | m₁ | f₁ | p₁ = ¼(1 − θ)² + ¼θ (1 − θ) + ¼θ² | p₆ | (¼θ (1 − θ) + ½θ²)/p1 | (¼θ (1 − θ))/p₆ |
| Aa | expressed | m₂ | f₂ | p₂ = ¼(1 − θ)² + θ (1 − θ) + ¼θ² | p₅ | (θ (1 − θ) + ½θ²)/p2 | (½θ²)/p5 |
| aa | expressed | m₃ | f₃ | p₃ = ¼(1 − θ)² + ¼θ (1 − θ) + ¼θ² | p₄ | (¼θ (1 − θ) + ½θ²)/p₃ | (¼θ (1 − θ))/p4 |
| AA | unexpressed | m₄ | f₄ | p₄ = ¼(1 − θ) | p₃ | (¼θ (1 − θ))/p4 | (¼θ (1 − θ) + ½θ²)/p3 |
| Aa | unexpressed | m₅ | f₅ | p₅ = ¼(1 − θ)² + ¼θ² | p₂ | (½θ²)/p5 | (θ (1 − θ) + ½θ²)/p2 |
| aa | unexpressed | m₆ | f₆ | p₆ = ¼θ (1 − θ) | p₁ | (¼θ (1 − θ))/p6 | (¼θ (1 − θ) + ½θ²)/p1 |

^{a}M and F denote the male and female offspring respectively.

where λ₁=θ/(1+θ), λ₂=θ(1+θ)/(1−θ+θ²), λ₃=1/(1−½θ), λ₄=θ/(1+2θ−2θ²), λ₅=θ/(1−2θ+2θ²), a=(m₁+f₆)/2n, b=(m₂+f₅)/2n, c=(m₃+f₄)/2n, d=(M₄+f₃)/2n, e=(m₅+f₂)/2n, g=(m₆+f₁)/2n, and where mᵢ and fᵢ are defined in Table 12.

LOD scores may be determined according to formula 41 above.

The system also computes direct counting data for locus pairs in case 0 (block 526). Using haplotype frequency data, the recombination frequencies and LOD scores are directly computed for each locus pair. Direct counting methods for determining recombination frequencies and LOD scores are known in the art.

Next, the computed indirect counting data and direct counting data are combined (block 528). Recombination frequencies and LOD scores based on both direct and indirect counting methods are combined to compute a single recombination frequency and LOD score for each locus pair.

Returning to FIG. 5A, the loci are ordered (block 508). The order loci functions split the loci into linkage groups and orders each linkage group, based on recombination frequencies and LOD scores previously computed.

FIG. 5E is a flowchart providing further details of the order loci processing of block 508. A system executing the method begins by determining linkage groups (block 530). All of the loci are divided into distinct linkage groups.

Next, for each linkage group the system computes Two-point Likelihoods (block 534) A likelihood is computed for each locus pair in the linkage group, this is used for ordering the loci. The most likely orders of the loci in the linkage group are computed using one of three different ordering methods, quick order (block 536), brute force order (block 538), or 3-point order (block 540). The most likely orders for the linkage group may then be placed in an output data stream (block 542). In addition, the most likely orders for the linkage groups, may be placed to an output data stream.

Next, a linkage map is computed for the most likely order for the linkage group and printed to an output file (block 544).

Returning to FIG. 5A, a system executing the invention may output additional data (block 510) In some embodiments, this additional data comprises pairwise data comprising pairwise recombination frequencies and LOD scores and locus info. In further embodiments, locus info comprising information about the informativeness of each locus is computed and placed on an output data stream.

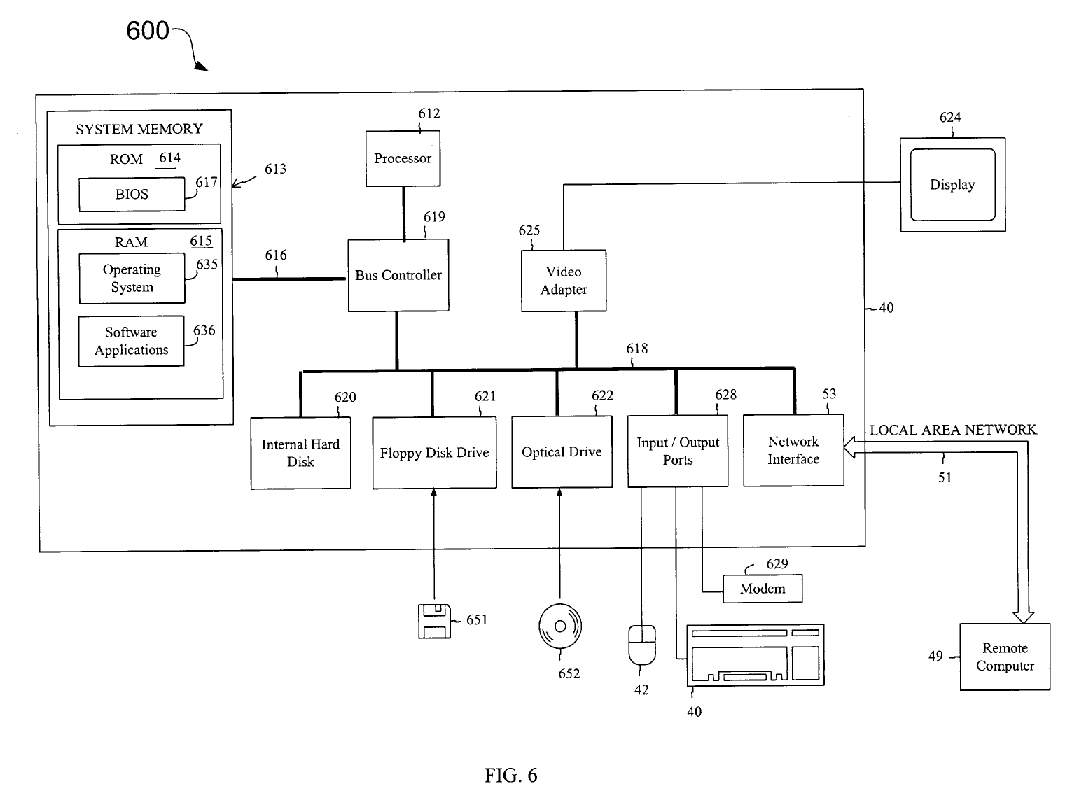

FIG. 6 is a diagram of the hardware and operating environment in conjunction with which embodiments of the invention maybe practiced. The description of FIG. 6 is intended to provide a brief, general description of suitable computer hardware and a suitable computing environment in conjunction with which the invention may be implemented. Although not required, the invention is described in the general context of computer-executable instructions, such as program modules, being executed by a computer, such as a personal computer or a server computer. Generally, program modules include routines, programs, objects, components, data structures, etc., that perform particular tasks or implement particular abstract data types.

Moreover, those skilled in the art will appreciate that the invention may be practiced with other computer system configurations, including hand-held devices, multiprocessor systems, microprocessor-based or programmable consumer electronics, network PCs, minicomputers, mainframe computers, and the like. The invention may also be practiced in distributed computing environments where tasks are performed by remote processing devices that are linked through a communications network. In a distributed computing environment, program modules may be located in both local and remote memory storage devices.

As shown in FIG. 6, the computing system 600 includes a processor. The invention can be implemented on computers based upon microprocessors such as the PENTIUM® family of microprocessors manufactured by the Intel Corporation, the MIPS® family of microprocessors from the Silicon Graphics Corporation, the POWERPC® family of microprocessors from both the Motorola Corporation and the IBM Corporation, the PRECISION ARCHITECTURE® family of microprocessors from the Hewlett-Packard Company, the SPARC® family of microprocessors from the Sun Microsystems Corporation, or the ALPHA® family of microprocessors from the Compaq Computer Corporation. Computing system 600 represents any personal computer, laptop, server, or even a battery-powered, pocket-sized, mobile computer known as a hand-held PC.

The computing system 600 includes system memory 613 (including read-only memory (ROM) 614 and random access memory (RAM) 615), which is connected to the processor 612 by a system data/address bus 616. ROM 614 represents any device that is primarily read-only including electrically erasable programmable read-only memory (EEPROM), flash memory, etc. RAM 615 represents any random access memory such as Synchronous Dynamic Random Access Memory.

Within the computing system 600, input/output bus 618 is connected to the data/address bus 616 via bus controller 619. In one embodiment, input/output bus 618 is implemented as a standard Peripheral Component Interconnect (PCI) bus. The bus controller 619 examines all signals from the processor 612 to route the signals to the appropriate bus. Signals between the processor 612 and the system memory 613 are merely passed through the bus controller 619. However, signals from the processor 612 intended for devices other than system memory 613 are routed onto the input/output bus 618.

Various devices are connected to the input/output bus 618 including hard disk drive 620, floppy drive 621 that is used to read floppy disk 651, and optical drive 622, such as a CD-ROM drive that is used to read an optical disk 652. The video display 624 or other kind of display device is connected to the input/output bus 618 via a video adapter 625.

A user enters commands and information into the computing system 600 by using a keyboard 40 and/or pointing device, such as a mouse 42, which are connected to bus 618 via input/output ports 628. Other types of pointing devices (not shown in FIG. 6) include track pads, track balls, joy sticks, data gloves, head trackers, and other devices suitable for positioning a cursor on the video display 624.

As shown in FIG. 6, the computing system 600 also includes a modem 629. Although illustrated in FIG. 6 as external to the computing system 600, those of ordinary skill in the art will quickly recognize that the modem 629 may also be internal to the computing system 600. The modem 629 is typically used to communicate over wide area networks (not shown), such as the global Internet. The computing system may also contain a network interface card 53, as is known in the art, for communication over a network.

Software applications 636 and data are typically stored via one of the memory storage devices, which may include the hard disk 620, floppy disk 651, CD-ROM 652 and are copied to RAM 615 for execution. In one embodiment, however, software applications 636 are stored in ROM 614 and are copied to RAM 615 for execution or are executed directly from ROM 614.

In general, the operating system 635 executes software applications 636 and carries out instructions issued by the user. For example, when the user wants to load a software application 636, the operating system 635 interprets the instruction and causes the processor 612 to load software application 636 into RAM 615 from either the hard disk 620 or the optical disk 652. Once software application 636 is loaded into the RAM 615, it can be used by the processor 612. In case of large software applications 636, processor 612 loads various portions of program modules into RAM 615 as needed.

The Basic Input/Output System (BIOS) 617 for the computing system 600 is stored in ROM 614 and is loaded into RAM 615 upon booting. Those skilled in the art will recognize that the BIOS 617 is a set of basic executable routines that have conventionally helped to transfer information between the computing resources within the computing system 600. These low-level service routines are used by operating system 635 or other software applications 636.

In one embodiment computing system 600 includes a registry (not shown) which is a system database that holds configuration information for computing system 600. For example, Windows® 95, Windows 98®, Windows® NT, Windows 2000® and Windows XP® by Microsoft maintain the registry in two hidden files, called USER.DAT and SYSTEM.DAT, located on a permanent storage device such as an internal disk.

### CONCLUSION

Systems and methods for performing linkage analysis using direct and indirect counting methods have been disclosed. The systems and methods described provide advantages over previous systems. For all the cases, direct and indirect counting typically yield the same results as maximum likelihood analysis. The inventive method of direct and indirect counting is therefore a useful addition or alternative to current methods available for linkage analysis including complex maximum likelihood analysis due to its mathematical simplicity and computational efficiency. When combined with the strategy of two-point analysis for linkage detection, the method of direct and indirect counting can provide rapid large scale joint linkage analysis of codominant and dominant loci, which is useful to facilitate mapping dominant loci using codominant markers and the map integration of codominant and dominant loci. The estimates of recombination frequencies from direct and indirect counting are the expected fraction of recombinants whether the estimates are within or out of the parameter space. This is helpful in interpreting the estimates in situations where the meanings of the estimates are not easily interpretable. For example, if a maximum likelihood using numerical maximization yielded an estimate out of the parameter space, the estimate itself could tell whether the problem was due to the algorithm of numerical maximization or due to a wrong model or sampling. A wrong inheritance model can result in a serious bias in estimating recombination frequencies (including estimates out of the parameter space) and such a bias can be evaluated conveniently using the method of direct and indirect counting.

The systems and methods of the present invention therefore provide simple solutions for linkage analysis to facilitate large scale joint linkage analysis with codominant and dominant loci, and for designing mapping experiments.

Although specific embodiments have been illustrated and described herein, it will be appreciated by those of ordinary skill in the art that any arrangement which is calculated to achieve the same purpose may be substituted for the specific embodiments shown. This application is intended to cover any adaptations or variations of the present invention.

The terminology used in this application is meant to include all of these environments. It is to be understood that the above description is intended to be illustrative, and not restrictive. Many other embodiments will be apparent to those of skill in the art upon reviewing the above description. Therefore, it is manifestly intended that this invention be limited only by the following claims and equivalents thereof.

We claim:

1. A method for performing genetic analysis, the method comprising:
receiving input data including family identification and genetic identifiers;
extracting statistics regarding the genetic identifiers; and
computing at least one recombination frequency for at least pair of loci by applying indirect counting to at least a subset of the statistics.

2. The method of claim 1 further comprising determining an inheritance case and wherein computing at least one recombination frequency uses the inheritance case to determine if indirect counting is to be applied to the statistics.

3. The method of claim 2 wherein the inheritance case comprises a biallelic codominant locus and a multiallelic codominant locus and wherein the at least one recombination frequency is computed substantially according to formula (1) or formula (2).

4. The method of claim 2 wherein the inheritance case comprises two biallelic codominant loci and wherein the at least one recombination frequency is computed substantially according to formula (7).

5. The method of claim 2 wherein the inheritance case comprises two biallelic codominant loci with a mixed linkage phase and wherein the at least one recombination frequency is computed substantially according to formula (9) or formula (10).

6. The method of claim 2 wherein the inheritance case comprises a multiallelic, codominant locus and a dominant/recessive locus and wherein the at least one recombination frequency is computed substantially according to formula (15) or formula (16).

7. The method of claim 2 wherein the inheritance case comprises a biallelic codominant locus and a dominant/recessive locus and wherein the at least one recombination frequency is computed substantially according to formula (21).

8. The method of claim 2 wherein the inheritance case comprises a biallelic codominant locus and a dominant/recessive locus with a mixed linkage phase and wherein the at least one recombination frequency is computed substantially according to formula (23) or formula (24).

9. The method of claim 2 wherein the inheritance case comprises two dominant/recessive loci with a coupling phase and wherein the at least one recombination frequency is computed substantially according to formula (29).

10. The method of claim 2 wherein the inheritance case comprises two dominant/recessive loci with a mixed phase and wherein the at least one recombination frequency is computed substantially according to formula (32).

11. The method of claim 2 wherein the inheritance case comprises two dominant/recessive loci with a repulsion phase and wherein the at least one recombination frequency is computed substantially according to formula (35).

12. The method of claim 2 wherein the inheritance case comprises a multiallelic codominant locus and a sex-linked locus and wherein the at least one recombination frequency is computed substantially according to formula (40).

13. The method of claim 2 wherein the inheritance case comprises a biallelic codominant locus and a sex-linked locus and wherein the at least one recombination frequency is computed substantially according to formula (42).

14. The method of claim 2 wherein the inheritance case comprises a biallelic codominant locus and a sex-linked locus with a mixed linkage phase and wherein the at least one recombination frequency is computed substantially according to formula (43).

15. The method of claim 1 wherein the genetic identifiers include genotype data.

16. The method of claim 1 wherein the genetic identifiers include phenotype data.

17. The method of claim 1 wherein the statistics include genotype frequencies.

18. The method of claim 1 wherein computing recombination frequencies includes applying an iterative computation to compute the at least one recombination frequency.

19. The method of claim 1 further comprising computing at least one LOD score for at least one locus by applying indirect counting to the at least one subset of the statistics.

20. The method of claim 1 further comprising identifying linked loci utilizing the at least one recombination frequency.

21. The method of claim 20 further comprising computing a locus order utilizing the at least one recombination frequency.

22. A computerized system for performing genetic analysis, the system comprising:
a data stream having locus information, said locus information including genetic identifiers; and
a linkage analysis program operable to perform the tasks of:
read the data stream;
extract statistics regarding the genetic identifiers; and
compute at least one recombination frequency for at least one pair of loci by applying indirect counting to at least a subset of the statistics.

24. The computerized system of claim 23 wherein the genetic identifiers include genotype data.

25. The computerized system of claim 23 wherein the genetic identifiers include phenotype data.

26. The computerized system of claim 23 wherein the statistics include genotype frequencies.

27. The computerized system of claim 23 wherein computing at least one recombination frequency includes applying an iterative computation to compute the at least one recombination frequency.

28. The computerized system of claim 23 wherein the linkage analysis program is further operable to compute at least one LOD score for at least one pair of loci by applying indirect counting to the at least one subset of the statistics.

29. The computerized system of claim 23 wherein the linkage analysis program is further operable to identify linked loci utilizing the recombination frequency.

30. The computerized system of claim 23 wherein the linkage analysis program is further operable to compute a locus order utilizing the at least one recombination frequency.

31. A computer-readable medium having computer executable instructions stored thereon for executing a method for performing genetic analysis, the method comprising:
receiving input data including family identification and genetic identifiers;
extracting statistics regarding the genetic identifiers; and
computing at least one recombination frequency for at least pair of loci by applying indirect counting to at least a subset of the statistics.

32. The computer-readable medium of claim 31 wherein the method further comprises determining an inheritance case and wherein computing at least one recombination frequency uses the inheritance case to determine if indirect counting is to be applied to the statistics.

33. The computer-readable medium of claim 31 wherein the inheritance case comprises a biallelic codominant locus and a multiallelic codominant locus and wherein the at least one recombination frequency is computed substantially according to formula (1) or formula (2).

34. The computer-readable medium of claim 32 wherein the inheritance case comprises two biallelic codominant loci and wherein the at least one recombination frequency is computed substantially according to formula (7).

35. The computer-readable medium of claim 32 wherein the inheritance case comprises two biallelic codominant loci with a mixed linkage phase and wherein the at least one recombination frequency is computed substantially according to formula (9) or formula (10).

36. The computer-readable medium of claim 32 wherein the inheritance case comprises a multiallelic, codominant locus and a dominant/recessive locus and wherein the at least one recombination frequency is computed substantially according to formula (15) or formula (16).

37. The computer-readable medium of claim 32 wherein the inheritance case comprises a biallelic codominant locus and a dominant/recessive locus and wherein the at least one recombination frequency is computed substantially according to formula (21).

38. The computer-readable medium of claim 32 wherein the inheritance case comprises a biallelic codominant locus and a dominant/recessive locus with a mixed linkage phase and wherein the at least one recombination frequency is computed substantially according to formula (23) or formula (24).

39. The computer-readable medium of claim 32 wherein the inheritance case comprises two dominant/recessive loci with a coupling phase and wherein the at least one recombination frequency is computed substantially according to formula (29).

40. The computer-readable medium of claim 32 wherein the inheritance case comprises two dominant/recessive loci with a mixed phase and wherein the at least one recombination frequency is computed substantially according to formula (32).

41. The computer-readable medium of claim 32 wherein the inheritance case comprises two dominant/recessive loci with a repulsion phase and wherein the at least one recombination frequency is computed substantially according to formula (35).

42. The computer-readable medium of claim 32 wherein the inheritance case comprises a multiallelic codominant locus and a sex-linked locus and wherein the at least one recombination frequency is computed substantially according to formula (40).

43. The computer-readable medium of claim 32 wherein the inheritance case comprises a biallelic codominant locus and a sex-linked locus and wherein the at least one recombination frequency is computed substantially according to formula (42).

44. The computer-readable medium of claim 32 wherein the inheritance case comprises a biallelic codominant locus and a sex-linked locus with a mixed linkage phase and wherein the at least one recombination frequency is computed substantially according to formula (43).

45. The computer-readable medium of claim 31 wherein the genetic identifiers include genotype data.

46. The computer-readable medium of claim 31 wherein the genetic identifiers include phenotype data.

47. The computer-readable medium of claim 31 wherein the statistics include genotype frequencies.

48. The computer-readable medium of claim 31 wherein computing recombination frequencies includes applying an iterative computation to compute the at least one recombination frequency.

49. The computer-readable medium of claim 31 wherein the method further comprises computing at least one LOD score for at least one locus by applying indirect counting to the at least one subset of the statistics.

50. The computer-readable medium of claim 31 wherein the method further comprises identifying linked loci utilizing the at least one recombination frequency.

51. The computer-readable medium of claim 50 further comprising computing a locus order utilizing the at least one recombination frequency.
